(19) United States
(12) Patent Application Publication
(10) Pub. No.: US 2023/0012071 A1
(43) Pub. Date: Jan. 12, 2023

(54) MODIFIED RELEASE PHARMACEUTICAL COMPOSITIONS OF RIOCIGUAT

(71) Applicant: ZYDUS LIFESCIENCES LIMITED, Ahmedabad (IN)

(72) Inventors: Kannan Essakimuthu MUTHAIYYAN, Ahmedabad (IN); Chetan Rajashekhara MURTHY, Ahmedabad (IN); Vimal Tulsidas KANERIA, Ahmedabad (IN); Nikalesh Ravindrabhai PATEL, Ahmedabad (IN)

(73) Assignee: ZYDUS LIFESCIENCES LIMITED, Ahmedabad (IN)

(21) Appl. No.: 17/782,237

(22) PCT Filed: Dec. 5, 2020
(86) PCT No.: PCT/IB2020/061558

§ 371 Date: Jun. 3, 2022

(30) Foreign Application Priority Data
Dec. 5, 2019 (IN) 201921050140

(51) Int. Cl. A61K 9/00; A61K 9/28; A61K 9/20; A61K 31/506

(52) U.S. Cl. CPC A61K 9/0004; A61K 9/2866; A61K 9/2054; A61K 31/506

(57) ABSTRACT

The present invention relates to modified release pharmaceutical compositions of riociguat or a pharmaceutically acceptable salt thereof. In particular, the compositions of the invention are stable, possess formulation characteristics and also provide extended therapeutically effective plasma levels over twenty four hour time period. The invention also relates to processes of preparing such compositions.

### FIELD OF THE INVENTION

The present invention relates to modified release pharmaceutical compositions of riociguat or a pharmaceutically acceptable salt thereof. In particular, the compositions of the invention are stable, possess formulation characteristics and also provide extended therapeutically effective plasma levels over twenty four hour time period. The invention also relates to processes of preparing such compositions.

### BACKGROUND OF THE INVENTION

Riociguat, 3,5-diamino-6-(2,3-dichlorophenyl)-1,2,4-triazine, has the chemical formula C₂₀HF₁₉FN₈O₂ and the following structural formula:

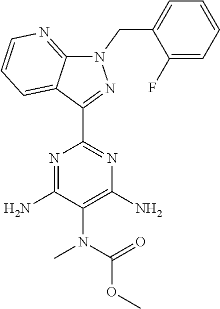 [SMILES: COC(=O)N(C)c1c(N)nc(-c2nn(Cc3ccccc3F)c3ncccc23)nc1N]

Riociguat is a stimulator of soluble guanylate cyclase (sGC), an enzyme in the cardiopulmonary system and the receptor for nitric oxide (NO) and useful for the treatment of pulmonary hypertension.

U.S. Pat. No. 7,173,037 discloses riociguat and its related compounds, along with their pharmaceutically acceptable salts.

Riociguat is approved in the US under the brand Adempass® as an immediate release tablet form with doses ranging from 0.5 mg to 2.5 mg administered TID (three times a day) for the treatment of chronic-thromboembolic pulmonary hypertension and pulmonary arterial hypertension.

Existing marketed tablet formulations of riociguat provide immediate release of the active ingredient once the tablet reaches the stomach. The absolute bioavailability of riociguat is high, about 94% and the peak plasma concentrations are observed within 1.5 hours following drug administration. Thus, administration of an immediate-release tablet can lead to greater frequency of adverse pharmacological events due to fast rate of absorption. Another disadvantage of the immediate release tablet dosage form is that the blood plasma concentration achieved with these tablets is cyclical, with peaks occurring after administration of the drug followed by troughs occurring before the next administration of the drug.

Therefore, there is an existing and continual need of a modified release pharmaceutical composition containing riociguat with reliable slower absorption over a targeted period of time to reduce fluctuations in blood plasma concentration of the drug during the dosing interval, which may address some of the concerns associated with the use of riociguat. Also, it may be possible to minimize the amount of drug required to achieve the desired therapeutic effect, which can improve the safety and tolerability profile of the drug.

There is a need to provide a modified release pharmaceutical composition of riociguat with reliable absorption over a targeted period of time and alongside possessing good formulation characteristics desired for bulk manufacturing of the product.

### SUMMARY OF THE INVENTION

In one general aspect, there is provided a modified release pharmaceutical composition comprising riociguat and one or more release retarding agents.

In another general aspect, there is provided a modified release pharmaceutical composition of riociguat suitable for once-a-day or twice-a-day administration.

Modified release of riociguat may be accomplished by use of matrix dosage forms, reservoir dosage forms, multiparticulate dosage forms, mucoadhesive dosage forms or osmotic dosage forms.

In another general aspect, there is provided a modified release pharmaceutical composition comprising a matrix of riociguat with one or more release retarding agents and one or more pharmaceutically acceptable excipients. The matrix of the dosage form remains substantially intact during the period of riociguat release. The matrix of the dosage form releases riociguat by diffusion and/or erosion.

In another general aspect, there is provided a modified release pharmaceutical composition comprising a core comprising riociguat, an optional barrier coating over the core, and at least one coating comprising one or more release retarding agents.

In another general aspect, there is provided a modified release pharmaceutical composition comprising—

(a) a sustained release component comprising plurality of particles comprising riociguat and one or more release retarding agents; and
(b) an immediate release component comprising riociguat coated over the sustained release component.

In another general aspect, there is provided a modified release pharmaceutical composition comprising a core comprising riociguat, one or more osmogens, and optionally one or more release retarding agents, and a semi-permeable coating. The semi-permeable coating comprises one or more water-insoluble polymers. The dosage form may deliver riociguat primarily by osmotic pressure.

In another general aspect, there is provided a stable modified release pharmaceutical composition comprising riociguat, one or more release retarding agents and optionally, one or more pharmaceutically acceptable excipients, wherein the composition is stable at 40° C. and 75% relative humidity for at least one month.

Embodiments of the modified release pharmaceutical composition may include one or more of the following features. For example, the pharmaceutically acceptable excipient may include one or more fillers, binders, disintegrants, glidants/lubricants/antiadherents, osmogens, plasticizers, and the like.

In another general aspect, there is provided a method of treating hypertension, which comprises orally administering to a patient a therapeutically effective amount of riociguat in the form of a modified release pharmaceutical composition comprising riociguat and one or more release retarding agents.

Representative modified release pharmaceutical compositions include a tablet, a capsule, granules, pellets, minitablets, a capsule filled with minitablets and/or pellets, or granules filled in a sachet.

The details of one or more embodiments of the invention are set forth in the description below. Other features, objects and advantages of the invention will be apparent from the description.

### DETAILED DESCRIPTION OF THE INVENTION

The inventors of the present invention have surprisingly found that it is possible to develop a stable modified release pharmaceutical composition of riociguat, which is suitable for once-a-day or twice-a-day administration with reliable absorption over a targeted period of time. The compositions according to the present invention also possess good formulation characteristics (such as flowability, compressibility, content uniformity etc.) desired for bulk manufacturing of the product.

The present invention relates to an oral modified release composition of riociguat for the treatment of pulmonary hypertension. Modified release of riociguat may be accomplished by use of matrix dosage forms, multiparticulate dosage forms, reservoir dosage forms, mucoadhesive dosage forms or osmotic dosage forms.

The term “riociguat” should be understood, unless otherwise indicated herein, to include any pharmaceutically acceptable form and salt of the compound. Riociguat may be present in crystalline or amorphous form. The pharmaceutical composition of the present invention comprises about 0.5 mg to about 7.5 mg of riociguat. Typically, the modified release composition of the present invention comprises from about 0.5% to 10% by weight of riociguat.

The term “about” as used herein means in reasonable vicinity of the stated numerical value, for example, plus or minus 10% or 5%.

The term “modified release pharmaceutical composition” as used hereinbefore and throughout the description includes a dosage form containing components providing extended release or combination of immediate release and extended release of the drug from the dosage form. The term “extended release” may be used alternatively as “sustained release”, “controlled release” or “delayed release”.

The term “component” as used hereinbefore and throughout the description refers to riociguat containing powder, particles, agglomerates, granules, pellets, minitablets, microcapsules, tablets, cores and coats/layers on thereof or any solid physical form known to the person skilled in the art.

The term “release retarding agent” as used hereinbefore and throughout the description refers to any material or substance that slows the release of the drug from the pharmaceutical composition. A release retarding agent may be a polymer or a non-polymer. In different delivery systems, the term “release retarding agent” may be used as different terminologies, for example, “an entraining agent” in osmotic dosage forms, “a mucoadhesive polymer” in mucoadhesive dosage forms etc.

The dosage forms of the present invention, in addition to the release retarding agents, may also include one or more pharmaceutically acceptable excipients like diluents, binders, disintegrants, glidants,/lubricants/antiadherents, osmogens, plasticizers, etc.

Examples of suitable diluents include, but not limited to, powdered sugar, calcium phosphate, calcium sulfate, microcrystalline cellulose, powdered cellulose, lactose, mannitol, kaolin, sodium chloride, dry starch, and sorbitol. The diluent may be present in an amount of from 5% to 90% by weight of the composition.

Examples of suitable binders include, but are not limited to, polyvinylpyrrolidone, xanthan gum, cellulose derivatives such as carboxymethylcellulose, methylcellulose, hydroxyethyl cellulose, hydroxypropyl cellulose, hydroxypropyl methylcellulose, gelatin, starch, and pregelatinized starch. The binder may be present in an amount of from 1% to 20% by weight of the composition.

Examples of suitable disintegrants include, but are not limited to, crosslinked polyvinylpyrrolidone, crosslinked sodium carboxymethyl cellulose, pregelatinized starch, low-substituted hydroxypropylcellulose and sodium starch glycolate. The disintegrant may be present in an amount of from 0.5% to 20% by weight of the composition.

Examples of suitable lubricants or glidants or antiadherents include, but are not limited to, talc, colloidal silicon dioxide, stearic acid, vegetable oil, calcium stearate, zinc stearate, and magnesium stearate. The lubricant may be present in an amount of from 0.1% to 10% by weight of the composition.

Examples of suitable osmogens include, but are not limited to, potassium chloride, magnesium sulfate, calcium sulfate, magnesium chloride, sodium chloride, lithium chloride, potassium sulfate, sodium carbonate, sodium sulfite, lithium sulfate, potassium chloride, sodium sulfate, d-mannitol, urea, sorbitol, inositol, raffinose, sucrose, glucose, hydrophilic polymers such as cellulose polymers, and mixtures thereof. The osmogen may be present in an amount of from 10% to 20% by weight of the composition.

Examples of suitable plasticizers include, but are not limited to, dibutyl phthalate, dibutyl sebacate, diethyl phthalate, dimethyl phthalate, triethyl citrate, benzyl benzoate, butyl and glycol esters of fatty acids, mineral oil, oleic acid, stearic acid, cetyl alcohol, stearyl alcohol, castor oil, corn oil, coconut oil, and camphor oil. The plasticizer may be present in an amount of from 0.01% to 20% by weight of the composition.

Other excipients that can be incorporated into the dosage form include, but are not limited to, preservatives, antioxidants, or any other excipient commonly used in the pharmaceutical industry.

### Matrix Dosage Forms:

In one embodiment, riociguat is embedded into an erodible or non-erodible polymeric matrix. An erodible matrix includes a matrix which is either erodible or swellable or dissolvable in aqueous medium in the presence or absence of an acid or base to ionize the polymeric matrix sufficiently to cause erosion or dissolution. When contacted with the aqueous medium, the erodible polymeric matrix absorbs water and forms an aqueous-swollen gel or “matrix” that entraps riociguat. The aqueous-swollen matrix gradually erodes, swells, disintegrates, disperses or dissolves in the environment of use, thereby controlling the release of riociguat to the environment of use.

Examples of polymers used for preparation of water-swellable, erodible, or soluble matrix include but not limited to, naturally occurring polysaccharides such as chitin, chitosan, dextran and pullulan; gum agar, gum arabic, gum karaya, locust bean gum, gum tragacanth, carrageenans, guar gum, xanthan gum and scleroglucan; starches such as dextrin and maltodextrin; hydrophilic colloids such as pectin; alginates such as ammonium alginate, sodium, potassium or calcium alginate, propylene glycol alginate; gelatin; collagen; and cellulosics such as methylethyl cellulose, carboxymethyl cellulose, carboxymethylethylcellulose, hydroxyethyl cellulose, hydroxypropyl cellulose, hydroxypropyl methylcellulose, hydroxypropyl methylcellulose phthalate, hydroxypropyl methylcellulose acetate succinate, hydroxypropyl methylcellulose acetate trimellitate, and ethylhydmxy ethylcellulose.

In a non-erodible matrix system, riociguat is distributed in an inert matrix. The drug is released by diffusion through the inert matrix. Examples of materials suitable for the inert matrix include, but not limited to, copolymers of ethylene and vinyl acetate, methyl acrylate-methyl methacrylate copolymers, polyvinyl chloride, and polyethylene; ethyl cellulose, cellulose acetate, crosslinked polyvinylpyrrolidone, and fatty compounds, such as carnauba wax, microcrystalline wax, and triglycerides.

The erodible or non-erodible polymeric release retardant may be present in an amount of from 10% to 70% by weight of the composition.

The modified release matrix dosage forms of the present invention may be generally prepared using standard techniques well known in the art. Typically, they may be prepared by dry blending riociguat, the release retarding agent, and other excipients followed by granulating the mixture using a solvent until proper granulation is obtained. The granulation may be done by methods known in the art. The wet granules may be dried in a fluid bed dryer, sifted and ground to appropriate size. Lubricating agents may be mixed with the dried granules to obtain the final dosage form. The release retarding agent may be used intragranular or extragranular or both.

Alternatively, the matrix dosage forms may be prepared using direct compression of a powdered or granular composition containing riociguat, alone or in combination with one or more release retarding agents, and one or more pharmaceutically acceptable excipients.

In another embodiment, a matrix multiparticulate dosage form comprises a plurality of riociguat containing particles, each particle comprising a mixture of riociguat with one or more release retarding agents selected to form a matrix capable of limiting the dissolution rate of the riociguat into an aqueous medium. The release retarding agents useful for this embodiment are generally water-insoluble materials such as waxes, cellulose, or other water-insoluble polymers. If needed, the matrix may optionally be formulated with water-soluble materials which can be used as binders or as permeability-modifying agents.

Examples of the release retarding agents useful for the manufacture of these dosage forms include microcrystalline cellulose, waxes such as paraffin, modified vegetable oils, carnauba wax, hydrogenated castor oil, beeswax, and the like, as well as synthetic polymers such as polyvinyl chloride, polyvinyl acetate, copolymers of vinyl acetate and ethylene, polystyrene, and the like. Water soluble binders or release retarding agents which can optionally be formulated into the matrix include water-soluble polymers such as hydroxypropyl cellulose, hydroxypropyl methylcellulose, methyl cellulose, polyvinylpyrrolidinone, polyethylene oxide, polyvinyl alcohol, xanthan gum, carrageenan, and other such natural and synthetic materials.

The matrix multiparticulates may be generally prepared by the extrusion/spheronization process. For this process, the riociguat may be wet-massed with a binder, extruded through a perforated plate or die, and placed on a rotating disk. The extrudates may be broken down into pieces which may be rounded into spheres, spheroids, or rounded rods on the rotating plate.

Alternatively, the matrix multiparticulates may be prepared by preparing wax granules. In this process, a desired amount of riociguat may be stirred with liquid wax to form a homogeneous mixture, cooled and then forced through a screen to form granules.

A further process for manufacturing matrix multiparticulates may involve use of an organic solvent to aid mixing of riociguat with the release retarding agent. Riociguat and release retarding agent may be combined with a solvent to form a paste, and then forced through a screen to form granules from which the solvent may be then removed.

Alternatively, riociguat and the release retarding agent may be combined with one or more solvents to completely dissolve the release retarding agent and the resulting solution (which may contain solid drug particles) may be spray dried to form the particulate dosage form. This technique may be preferred when the release retarding agent is a high molecular weight synthetic polymer such as a cellulose ether or a cellulose ester. Solvents typically employed for the process may include acetone, ethanol, isopropanol, ethyl acetate, and mixtures of two or more.

In another embodiment, the matrix muhiparticulates may be formed by the melt spray congeal process. The melt-congeal core may comprise a release retarding agent.

The release retarding agent may be present in the core from at least 10% to 70% by weight based on the mass of the uncoated core.

The release retarding agent for melt spray congeal method is selected from the group consisting of waxes, long chain alcohols, fatty acid esters, glycolized fatty acid esters, phosphoglycerides, polyoxyethylene alkyl ethers, long chain carboxylic acids, sugar alcohols, and mixtures thereof. Exemplary release retarding agents include highly purified forms of waxes, such as carnauba wax, white and yellow beeswax, ceresin wax, microcrystalline wax, and paraffin wax; long-chain alcohols, such as stearyl alcohol, cetyl alcohol and polyethylene glycol: fatty acid esters (also known as fats or glycerides), such as isopropyl palmitate, isopropyl myristate, glyceryl monooleate, glyceryl monostearate, glyceryl palmitostearate, mixtures of mono-, di-, and trialkyl glycerides, including mixtures of glyceryl mono-, di-, and tribehenate, glyceryl tristearate, glyceryl tripalmitate and hydrogenated vegetable oils, including hydrogenated cottonseed oil; glycolized fatty acid esters, such as polyethylene glycol stearate and polyethylene glycol distearate; polyoxyethylene alkyl ethers; polyethoxylated castor oil derivatives; long-chain carboxylic acids such as stearic acid; and sugar alcohols such as mannitol and erythritol.

The multiparticulates are made via a melt-congeal process comprising the steps: (a) forming a molten mixture comprising riociguat and a release retarding agent; (b) delivering the molten mixture of step (a) to an atomizing means to form droplets from the molten mixture; and (c) congealing the droplets from step (b) to form multiparticulates.

The multiparticulates may also be mixed or blended with one or more pharmaceutically acceptable excipients to form a suitable dosage form. Suitable dosage forms may include tablets, capsules, sachets, oral powder for constitution, and the like.

Following the formation of the melt spray congeal multiparticulates, the multiparticulates may optionally be coated with any conventional coating, such as a protective film coating, a coating to provide sustained release of the drug, or a coating to provide taste-masking.

The coating may include conventional plasticizers, and other excipients such as anti-tack agents, glidants, etc. The coating may be applied by conventional instruments, such as by pan coaters, fluidized bed coaters such as top-spray, tangential-spray or bottom-spray.

### Osmotic Dosage Forms

Riociguat is incorporated into osmotic delivery devices or osmotic pumps. Osmotic pumps comprise a core containing an osmotically effective composition surrounded by a semipermeable coating. The semipermeable coating is permeable to water, but solutes dissolved in water typically cannot readily diffuse through the coating.

In one embodiment of the present invention, riociguat is incorporated into a bilayer tablet core comprising first layer comprising riociguat, one or more release retarding agents and one or more osmogens, and second layer comprising one or more water-swellable polymers, optionally one or more osmogens. The bilayer tablet core is surrounded by a semi-permeable coating which contains one or more openings or orifices which are manufactured into the dosage form through laser drilling. The release retarding agent used in this type of dosage form is also mentioned as an entraining agent. An entraining agent entrains the drug so as to aid in the delivery of the drug through one or more orifices.

The release retarding agent or the entraining agent may be a single material or a mixture of materials. Non-crosslinked polyethylene oxide may be used as the entraining agent. Examples of other suitable entraining agents include, but not limited to, hydroxypropyl cellulose, hydroxypropyl methylcellulose, methylcellulose, hydroxyethyl cellulose and polyvinylpyrrolidone, as well as mixtures of these polymers with polyethylene oxide. The amount of the release retarding agent or the entraining agent present in the composition may range from 15% to 95% by weight of the composition.

Examples of suitable water-swellable polymers include, but not limited to, sodium starch glycolate, croscarmellose sodium, poly(hydroxyalkyl methacrylate) having a molecular weight of from 30,000 to 5.000,000; kappa carrageenan, polyvinylpyrrolidone having molecular weight of from 10,000 to 360.000; anionic and cationic hydrogels; polyelectrolyte complexes; poly(vinyl alcohol) having a low acetate residual, cross-linked with glyoxal; a mixture of methyl cellulose; cross-linked agar and carboxymethyl cellulose; water swellable polymers of N-vinyl lactams, and the like. Such polymers may be present in an amount of from 0.5% to 20% by weight of the composition.

Examples of suitable materials for the semipermeable coating include, but are not limited to, cellulose acylate, cellulose diacylate, cellulose triacylate, cellulose acetate, cellulose diacetate, cellulose triacetate, agar acetate, amylose triacetate, beta glucan acetate, acetaldehyde dimethyl acetate, cellulose acetate ethyl carbamate, polyamides, polyurethanes, sulfonated polystyrenes, cellulose acetate phthalate, cellulose acetate methyl carbamate, cellulose acetate succinate, cellulose acetate dimethyl aminoacetate, cellulose acetate ethyl carbamate, cellulose acetate chloracetate, cellulose dipalmitate, cellulose dioctanoate, cellulose dicaprylate, cellulose dipentanlate, cellulose acetate valerate, cellulose acetate succinate, cellulose propionate succinate, methyl cellulose, cellulose acetate p-toluene sulfonate, cellulose acetate butyrate, cross-linked poly(sodium styrene sulfonate), poly(vinylbenzyltrimethyl ammonium chloride), and the like.

The semipermeable membrane coating on the tablet, is present at a weight corresponding to from 2% to 30% of the weight of the tablet core.

The expression “orifice” as used herein includes one or more apertures which have been bored through the semipermeable membrane by mechanical procedures. Alternatively, it may be formed by incorporating an erodible element, such as a gelatin plug, in the semipermeable membrane.

The osmotic pumps of the present invention may be manufactured by standard techniques. For example, in one embodiment, the drug and other excipients that may be housed in one area of the compartment adjacent to the passageway, may be pressed into a solid possessing dimension that corresponds to the internal dimensions of the area of the compartment the drug will occupy, or the drug and other excipients along with a solvent may be mixed into a solid or semisolid form by conventional methods such as ball-milling, calendaring, stirring or roll-milling, and then pressed into a preselected shape. Next, a layer of a hydrophilic polymer may be placed in contact with the layer of the drug in a like manner, and the two layers may be surrounded with a semipermeable wall. The layering of drug formulation and hydrophilic polymer may be fabricated by conventional two-layer press techniques. The wall may be applied by molding, spraying or dipping the pressed layers into a wall forming material or a release retarding agent.

Alternatively, the core of the osmotic dosage form may be tri-layer tablet core. The tri-layer tablet core comprises first layer comprising riociguat and other excipients, second layer comprising higher amount of riociguat and other excipients and third layer which is a push layer and is devoid of riociguat. In the tri-layer osmotic dosage form, drug is successively released, in a controlled manner, from the first drug-containing layer and then from the second drug-containing layer at a controlled manner to provide ascending release rate for an extended time period.

### Reservoir Dosage Forms

In another embodiment of the present invention, there is provided a composition comprising a core comprising riociguat and a release retarding coating on one or more of the outer surfaces of the composition for example a tablet or a bead. A conventional immediate release compression tablet may be at least partially coated by a coating comprising a release retarding agent or alternatively, a pharmaceutically acceptable bead is used in which the riociguat is incorporated and then the head is at least partially coated by a coating comprising a release retarding agent. In some embodiments, additional barrier coating may be present in between the core and the coating.

The coating may, for example, be composed of polymers which are either substantially or completely impermeable to water or aqueous media, or are slowly erodable in water or aqueous media or biological liquids and/or which swell in contact with water or aqueous media or biological liquids. The coating may also include conventional binders, plasticizers, fillers, lubricants, colorants, compression aids, etc.

Examples of suitable polymers for the coating include, but not limited to, acrylates, methacrylates, copolymers of acrylic acid or its esters, celluloses and derivatives thereof such as ethylcelluloses, cellulose acetate propionate, polyethylenes and polyvinyl alcohol etc.

The amount of the polymer present in the composition may range from 1% to 30% by weight of the composition.

### Multiparticulate Dosage Forms

There is provided a composition comprising multiparticulates wherein the particles are coated with one or more release retarding agents to provide modified release of riociguat. The multiparticulate particles comprise riociguat and one or more excipients. The size of individual particles is generally between about 50 micron and about 2 mm, although beads of a size outside this range may also be useful.

The multiparticulates composition may be prepared using techniques known to those skilled in the art, including, but not limited to, the techniques of extrusion and spheronization, wet granulation, fluid bed granulation, and rotary bed granulation. In addition, the multiparticulates may also be prepared by building the riociguat composition (drug plus excipients) up on a seed core, such as a non-pareil seed, by a drug-layering technique such as powder coating or by applying the riociguat composition by spraying a solution or dispersion of riociguat in an appropriate binder solution onto seed cores in a fluidized bed such as a wurster coater or a rotary processor.

In another embodiment of the present invention, there is provided a pharmaceutical composition that incorporates immediate release beads and controlled release beads capable of release of riociguat over approximately 10 hours to approximately 24 hours. The pharmaceutical composition of this embodiment generally comprises immediate release riociguat beads in an amount ranging from about 10% to about 50% by weight of the total composition weight and controlled release riociguat beads in an amount ranging from about 50% to about 90% by weight of the total composition weight.

These multiparticulates may be compressed into a tablet dosage form or alternatively, these multiparticulates may be filled into a capsule or a sachet.

### Mucoadhesive Dosage Forms

In another embodiment, there is provided a modified release pharmaceutical composition of riociguat comprising one or more release retarding agents. The release retarding agents used in this type of dosage form are also mentioned as mucoadhesive polymers.

A mucoadhesive polymer is a natural or synthetic polymer capable of adhering to the mucous lining of the gastrointestinal tract. The mucoadhesive polymer may be applied as a coating or as a part of the drug delivery matrix. Mucoadhesive polymers are included in the composition to improve gastrointestinal retention via adherence of the composition to the walls of the GI tract.

Examples of suitable mucoadhesive polymers include, but not limited to, polycarbophil (polyacrylic acid cross-linked with divinylglycol), chitosan, dextran sodium, poly-L-aspartic acid, polystyrene sulfonic acid, polyvinyl sulfate, polyglutamic acid, bovine serum albumin. ficoll, acidic (high isoelectric point) gelatin, polybrene, polyvinyl methyl imidazole, polygalactosamine, polyquaternary compounds, prolamine, polyimine, diethylaminoethyldextran, polyvinylpyridine, polyethylene pyrrolidone, polythiodiethyl aminomethylethylene, polyhistidine, poly-p-aminostyrene, polyoxethane, copolymethacrylates, polyamidoamines, and cationic starches.

The mucoadhesive polymer may be present in an amount of from 0.1% to 20% by weight of the composition.

The mucoadhesive composition may be in the form of a tablet, capsule, multiparticulate composition, or osmotic device.

The mucoadhesive composition of the present invention may be prepared by mixing riociguat, the mucoadhesive polymer, and other excipients followed by wet granulation, direct compression, extrusion, or spheronization method as described earlier.

In another embodiment of the present invention, there is provided a multilayer solid composition comprising (a) an immediate release first layer comprising riociguat and at least one pharmaceutically acceptable excipient, and (b) a sustained release second layer comprising riociguat, wherein the immediate release layer and the sustained release layer are optionally separated by a layer of excipient containing no drug. In forming the multilayer tablet, the layers may be prepared by conventional wet granulation or dry granulation (compaction) techniques, or any other technique known in the art. The layers may then be compressed and combined to form a multilayer tablet employing conventional multilayer tableting equipment. The sustained release layer contains one or more release retarding polymers as mentioned above for sustained release matrix systems. Additionally, all the layers may optionally contain one or more pharmaceutically acceptable excipients.

In another embodiment, there is provided a stable modified release pharmaceutical composition comprising riociguat, one or more release retarding agents and one or more pharmaceutically acceptable excipients.

The term “stable” as used herein means retaining substantially the same properties and characteristics of the active ingredient throughout its period of storage and use that it possessed at the time of its manufacture, such that the composition provides substantially the same therapeutic benefit to the patient over the period of time that the composition is stored and delivered.

In another embodiment, the composition described herein retains at least 80% of the potency of riociguat after storage at 40° C. and 75% relative humidity for at least 1 month, for example, for 3 months, 6 months, 12 months, 24 months etc.

In another embodiment, the present invention provides a modified release pharmaceutical composition of riociguat, wherein the composition releases not more than 15% of riociguat in 1 hour, and not less than 70% of riociguat in 16 hours, when subjected to a test medium comprising 900 mL of 0.05M pH 6.8 potassium phosphate buffer with 0.1% SLS at 37° C. in a standard USP rotating paddle apparatus at 75 rpm.

In an alternate embodiment, the composition releases not more than 10% of riociguat in 1 hour, and not less than 75% of riociguat in 16 hours, when subjected to a test medium comprising 900 mL of 0.05M pH 6.8 potassium phosphate buffer with 0.1% SLS at 37° C. in a standard USP rotating paddle apparatus at 75 rpm.

The present invention is illustrated by the following examples which are provided to be exemplary of the invention and do not limit the scope of invention.

### Example 1: Extended Release Osmotic Tablet

| | | |
|---|---|---|
| Sr. No. | Ingredients | mg/tablet |

Drug layer

| | | |
|---|---|---|
| 1 | Riociguat | 2.50 |
| 2 | Polyethylene oxide | 123.65 |
| 3 | Povidone | 22.50 |
| 4 | Magnesium stearate | 1.35 |
| 5 | Ethanol | Q.S. |

Push layer

| | | |
|---|---|---|
| 6 | Polyethylene oxide | 72.83 |
| 7 | Povidone | 14.67 |
| 8 | Potassium chloride | 10.00 |
| 9 | Magnesium stearate | 1.50 |
| 10 | Ferric oxide red | 1.00 |
| 11 | Ethanol | Q.S. |

Semipermeable coating

| | | |
|---|---|---|
| 12 | Cellulose acetate | 31.50 |
| 13 | Polyethylene glycol | 3.50 |
| 14 | Acetone | Q.S. |
| 15 | Purified Water | Q.S. |

### Procedure:

Granules for the preparation of the drug layer were prepared by granulating a powder mixture of riociguat, polyethylene oxide and povidone using a binder solution containing povidone dissolved in ethanol in a rapid mixer granulator. The prepared granules were dried in a fluid bed dryer and the dried granules were sifted to get appropriate dimension granules which were lubricated with magnesium stearate.

Granules for the preparation of the push layer were prepared by granulating a powder mixture of polyethylene oxide, povidone, potassium chloride and ferric oxide red using a binder solution containing povidone dissolved in ethanol using a rapid mixer granulator. The prepared granules were dried in a fluid bed dryer and the dried granules were sifted to get appropriate dimensions which were further lubricated with magnesium stearate.

The granules prepared in the above steps for the drug layer and the push layer were compressed using a bilayer compression machine with appropriate tooling to provide a bilayer core tablet.

A semipermeable membrane was coated surrounding the bilayer core tablet by spraying a solution of cellulose acetate and polyethylene glycol in acetone/water mixture.

A suitable orifice having diameter of around 0.6 t 0.3 mm and depth of 0.5*0.3 mm for the drug release was drilled through the semipermeable membrane at the narrow end of the compartment proximate to the drug layer.

Dissolution data was determined using in vitro dissolution testing apparatus using dissolution conditions given below:

Dissolution Conditions: 900 mL, pH 6.8 pH phosphate
buffer + 0.1% SLS, paddle with a sinker, speed - 75 rpm

| Time in | Cumulative percentage |
|---|---|
| hour | of drug release |
| 1 | 0.4 |
| 2 | 1.9 |
| 3 | 11.7 |
| 4 | 22.1 |
| 6 | 39.6 |
| 8 | 55.0 |
| 12 | 77.6 |
| 16 | 88.9 |
| 24 | 90.1 |

### Example 2: Extended Release Matrix Tablet

| Sr. No. | Ingredients | mg/tablet |
|---|---|---|
| 1 | Riociguat | 2.50 |
| 2 | Hypromellose | 22.50 |
| 3 | Microcrystalline cellulose | 123.50 |
| 4 | Magnesium stearate | 1.50 |
| 5 | Purified water | Q.S. |

Granules were prepared by granulating a powder mixture of riociguat, microcrystalline cellulose, and hypromellose, using a binder solution containing hypromcllosc dissolved in water using a rapid mixer granulator. The prepared granules were dried and lubricated with magnesium stearatc and compressed using a compression machine with appropriate tooling to provide a tablet.

### Example 3: Extended Release Reservoir Tablets

| | | |
|---|---|---|
| Sr. No. | Ingredients | mg/tablet |

Core tablet

| | | |
|---|---|---|
| 1 | Riociguat | 2.500 |
| 2 | Lactose monohydrate | 110.800 |
| 3 | Microcrystalline cellulose | 27.700 |
| 4 | Povidone | 7.500 |
| 5 | Magnesium stearate | 1.500 |
| 6 | Purified water | Q.S. |

Coating

| | | |
|---|---|---|
| 7 | Ethyl cellulose | 13.088 |
| 8 | Triethyl citrate | 1.640 |
| 9 | Hypromellose | 3.272 |
| 10 | Methylene chloride | Q.S. |
| 11 | Isopropyl alcohol | Q.S. |

Granules were prepared by granulating a powder mixture of riociguat, microcrystalline cellulose, and lactose monohydrate, using a binder solution containing povidone dissolved in water. The granules were dried and lubricated with magnesium stearate and compressed using a compression machine with appropriate tooling to provide tablets.

The tablets were coated by spraying a solution of ethyl cellulose, hypromellose and triethyl citrate in a solvent mixture of methylene chloride and isopropyl alcohol.

While the present invention has been described in terms of its specific embodiments, certain modification and equivalents will be apparent to those skilled in the art and are intended to include within the scope of the present invention.

We claim:

1. A modified release pharmaceutical composition comprising riociguat and one or more release retarding agents.

2. The pharmaceutical composition according to claim 1, wherein the composition comprises about 0.5 mg to about 7.5 mg of riociguat.

3. The pharmaceutical composition according to claim 1, wherein the release retarding agent is selected from the group consisting of water swellable polymer, water-insoluble polymer and non-polymeric material.

4. The pharmaceutical composition according to claim 1, wherein the composition comprises:
a. a core comprising the riociguat, one or more osmogens, and optionally one or more release retarding agents; and
b. a semi-permeable coating comprising one or more release retarding agent.

5. The pharmaceutical composition according to claim 4, wherein the release retarding agent is present in an amount of from 15% to 95% by weight of the composition.

6. The pharmaceutical composition according to claim 4, wherein the osmogen is present in an amount of from 2% to 55% by weight of the composition.

7. The pharmaceutical composition according to claim 4, wherein the release retarding agent in the semi-permeable coating is a water-insoluble polymer.

8. The pharmaceutical composition according to claim 7, wherein the water-insoluble polymer is present in an amount of from 2% to 30% by weight of the composition.

9. The pharmaceutical composition according to claim 4, wherein the core is a bilayer tablet comprising first layer comprising riociguat, one or more release retarding agents and one or more osmogens; and a second layer comprising a water-swellable polymer.

10. The pharmaceutical composition according to claim 9, wherein the water-swellable polymer is present in an amount of from 0.5% to 20% by weight of the composition.

11. The pharmaceutical composition according to claim 4, wherein the core comprises riociguat, polyethylene oxide and potassium chloride.

12. The pharmaceutical composition according to claim 1, wherein the riociguat is embedded in a matrix of one or more release retarding agents.

13. The pharmaceutical composition according to claim 12, wherein the release retarding agent is present in an amount of from 10% to 70% by weight of the composition.

14. The pharmaceutical composition according to claim 1, wherein the composition comprises:
a. a core comprising riociguat;
b. an optional barrier coating over the core; and
c. at least one coating comprising one or more release retarding agents.

15. The pharmaceutical composition according to claim 14, wherein the release retarding agent is present in an amount of from 1% to 30% by weight of the composition.

16. The pharmaceutical composition according to claim 1, wherein the composition is in the form of a tablet, a capsule, granules, pellets, mini-tablets, a capsule filled with mini-tablets and/or pellets, or granules filled in a sachet.

17. The pharmaceutical composition according to claim 1, wherein the composition is suitable for once-a-day administration.

18. The pharmaceutical composition according to claim 1, wherein the composition is suitable for twice-a-day administration.

19. The pharmaceutical composition according to claim 1, wherein the composition retains at least 80% of the potency of the riociguat in the composition after storage for at least one month at 40° C. and 75% relative humidity.

20. The pharmaceutical composition according to claim 1, wherein the composition releases riociguat not more than 15% in 1 hour, and not less than 70% in 16 hours, when subjected to a test medium comprising 900 mL of 0.05M pH 6.8 potassium phosphate buffer with 0.1% SLS at 37° C. in a standard USP rotating paddle apparatus at 75 rpm.
